SEQUENCE LISTING (10 sequences)

SEQ ID NO: 1 - protein, 5 aa, Mus musculus
RYWMH

SEQ ID NO: 2 - protein, 17 aa, Mus musculus
AIYPGNSDTSYNQKFEG

SEQ ID NO: 3 - protein, 8 aa, Mus musculus
DYGYYFDF

SEQ ID NO: 4 - protein, 10 aa, Mus musculus
SASSSISYMQ

SEQ ID NO: 5 - protein, 7 aa, Mus musculus
DTSKLAS

SEQ ID NO: 6 - protein, 7 aa, Mus Musculus
HQRSSYT

SEQ ID NO: 7 - DNA, 492 bp, Mus musculus
  CDS (1)...(45): Leader sequence of the immunoglobulin heavy chain variable domain
atggaatgtaactggatacttccttttattctgtcggtaatttcaggtaaggggctcaccagttccatatctgaaagaggatacagggtctgaagtgacaatgacatctactctgctgttctctccacaggggtctactcagaggttcagctccagcagtctgggactgtgctggctaggcctggggcttccgtgaagatgtcctgcaaggcttctggctacagctttaccaggtactggatgcactggataaaacagaggcctggacagggtctagaatggattggtgctatttatcctggaaatagtgatactagttacaaccagaagttcgagggcaaggccaaactgactgcagtcacatccgccagcactgcctacatggagctcagcagcctgacacatgaggactctgcggtctattactgttcaagagactacggctactactttgacttctggggccaaggcaccactctcacagtctcctca

SEQ ID NO: 8 - protein, 136 aa, Mus musculus
MECNWILPFILSVISGVYSEVQLQQSGTVLARPGASVKMSCKASGYSFTRYWMHWIKQRPGQGLEWIGAIYPGNSDTSYNQKFEGKAKLTAVTSASTAYMELSSLTHEDSAVYYCSRDYGYYFDFWGQGTTLTVSS

SEQ ID NO: 9 - DNA, 555 bp, Mus musculus
  CDS (1)...(48): Leader sequence of the immunoglobulin light chain variable domain
atggattttcaggtgcagattttcagcttcctgctaatcagtgcctcaggtaacagagggcagggaatttgagatcagaatccaaccaaaattattttccctggggaatttgagtctaaaatacagtttttttttctttttcttcatctgaatgttgggtggtataaaattatttttgtttctctatttctactaatccctttctctctattttgcttttttctagtcatactgtccagaggacaaattgttctcacccagtctccagcaatcatgtctgcatctccaggggagaaggtcaccatgacctgcagtgccagctcaagtataagttacatgcagtggtaccagcagaagccaggcacctcccccaaaagatggatttatgacacatccaaactggcttctggagtccctgctcgcttcagtggcagtgggtctgggacctcttattctctcacaatcagcagcatggaggctgaagatgctgccacttattactgccatcagcggagtagttacacgttcggaggggggaccaaactggaaataaaa

SEQ ID NO: 10 - protein, 126 aa, Mus Musculus
MDFQVQIFSFLLISASVILSRGQIVLTQSPAIMSASPGEKVTMTCSASSSISYMQWYQQKPGTSPKRWIYDTSKLASGVPARFSGSGSGTSYSLTISSMEAEDAATYYCHQRSSYTFGGGTKLEIK

(19) United States
(12) Patent Application Publication
(10) Pub. No.: US 2005/0226872 A1
(43) Pub. Date: Oct. 13, 2005

(54) Use of cd25 binding molecules in the treatmnet of inflammatory diseases of the gastrointestinal tract

(76) Inventors: Hansjorg Adam, Regensburg (DE); Lothar Farber, Heroldsberg (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/240,353

(22) PCT Filed: Mar. 28, 2001
(86) PCT No.: PCT/EP01/03541

§ 371 Date: Sep. 30, 2002

(30) Foreign Application Priority Data
Mar. 30, 2000 (GB) 0007911.1

(51) Int. Cl. A61K 39/395; C07K 16/28

(52) U.S. Cl. 424/144.1; 530/388.22

(57) ABSTRACT

Use of a CD25 binding molecule which comprises at least one antigen binding site comprising at least one domain which comprises in sequence, the hypervariable regions CDR1, CDR2 and CDR3; said CDR1 having the amino acid sequence Arg-Tyr-Trp-Met-His (SEQ ID NO:1), said CDR2 having the amino acid sequence Ala-Ile-Tyr-Pro-Gly-Asn-Ser-Asp-Thr-Ser-Tyr-Asn-Gln-Lys-Phe-Glu-Gly (SEQ ID NO:2), and said CDR3 having the amino acid sequence Asp-Tyr-Gly-Tyr-Tyr-Phe-Asp-Phe (SEQ ID NO:3), in the treatment of inflammatory disease of the gastro-intestinal tract.

### The invention is directed to the use of a CD25 binding molecule in the treatment of inflammatory diseases of the gastro-intestinal (GI) tract.

More specifically the present invention provides in a first aspect the use of a CD25 binding molecule which comprises at least one antigen binding site comprising at least one domain which comprises in sequence, the hypervariable regions CDR1, CDR2 and CDR3; said CDR1 having the amino acid sequence Arg-Tyr-Trp-Met-His, said CDR2 having the amino acid sequence Ala-Ile-Tyr-Pro-Gly-Asn-Ser-Asp-Thr-Ser-Tyr-Asn-Gln-Lys-Phe-Glu-Gly, and said CDR3 having the amino acid sequence Asp-Tyr-Gly-Tyr-Tyr-Phe-Asp-Phe; or direct equivalents thereof in the treatment of inflammatory diseases of the GI tract.

Treatment of inflammatory diseases of the GI tract includes control or amelioration of the disease and/or its sequellae, e.g. symptoms, as well as control or amelioration of aetiological components. Treatment also includes suppression of clinical relapse.

Inflammatory diseases of the GI tract include chronic inflammatory bowel diseases, such as Irritable Bowel Syndrome (IBS), Crohn's disease, ulcerative colitis and inflammatory intestinal disease and other inflammatory GI disorders and inflammatory diseases of the GI tract.

By “CD25 binding molecule” is meant any molecule capable of binding to the CD25 antigen either alone or associated with other molecules to form high affinity IL-2 receptors.

Preferably a CD25 binding molecule is used comprising at least one antigen binding site comprising:
a) a first domain comprising in sequence the hypervariable regions CDR1, CDR2 and CDR3; said CDR1 having the amino acid sequence Arg-Tyr-Trp-Met-His, said CDR2 having the amino acid sequence Ala-Ile-Tyr-Pro-Gly-Asn-Ser-Asp-Thr-Ser-Tyr-Asn-Gln-Lys-Phe-Glu-Gly, and said CDR3 having the amino acid sequence Asp-Tyr-Gly-Tyr-Tyr-Phe-Asp-Phe and,
b) a second domain comprising in sequence the hypervariable regions CDR1′, CDR2′ and CDR3′, said CDR1′ having the amino acid sequence Ser-Ala-Ser-Ser-Ser-Ile-Ser-Tyr-Met-Gln, said CDR2′ having the amino acid sequence Asp-Thr-Ser-Lys-Leu-Ala-Ser, and said CDR3′ having the amino acid sequence His-Gln-Arg-Ser-Ser-Tyr-Thr; or direct equivalents thereof.

Unless otherwise indicated, any polypeptide chain is herein described as having an amino acid sequence starting at the N-terminal extremity and ending at the C-terminal extremity.

When the antigen binding site comprises both the first and second domains, these may be located on the same polypeptide molecule or, preferably, each domain may be on a different chain, the first domain being part of an immunoglobulin heavy chain or fragment thereof and the second domain being part of an immunoglobulin light chain or fragment thereof.

Accordingly, the invention also provides the use of a CD25 binding molecule which comprises at least one antigen binding site comprising either a first domain having an amino acid sequence identical or substantially identical to that shown in Seq. Id. No. 1 in EP 449,769, the content of which is incorporated herein by reference, starting with amino acid at position 1 and ending with amino acid at position 117 or a first domain as described above and a second domain having an amino acid sequence identical or substantially identical to that shown in Seq. Id. No. 2 in EP 449,769, the contents of which is herein incorporated by reference, starting with amino acid at position 1 and ending with amino acid at position 104 in the treatment of inflammatory diseases of the GI tract.

A more preferred CD25 binding molecule for use in accordance with the invention is selected from a chimeric anti-CD25 antibody which comprises at least
a) one immunoglobulin heavy chain or fragment thereof which comprises (i) a variable domain comprising in sequence the hypervariable regions CDR1, CDR2 and CDR3 and (ii) the constant part or fragment thereof of a human heavy chain; said CDR1 having the amino acid sequence Arg-Tyr-Trp-Met-His, said CDR2 having the amino acid sequence Ala-Ile-Tyr-Pro-Gly-Asn-Ser-Asp-Thr-Ser-Tyr-Asn-Gln-Lys-Phe-Glu-Gly, and said CDR3 having the amino acid sequence Asp-Tyr-Gly-Tyr-Tyr-Phe-Asp-Phe and
b) one immunoglobulin light chain or fragment thereof which comprises (i) a variable domain comprising in sequence the hypervariable regions CDR1′, CDR2′ and CDR3′ and (ii) the constant part or fragment thereof of a human light chain; said CDR1′ having the amino acid sequence Ser-Ala-Ser-Ser-Ser-Ile-Ser-Tyr-Met-Gln, said CDR2′ having the amino acid sequence Asp-Thr-Ser-Lys-Leu-Ala-Ser, and said CDR3′ having the amino acid sequence His-Gln-Arg-Ser-Ser-Tyr-Thr;
   and direct equivalents thereof.

Alternatively, a CD25 binding molecule for use in accordance with the invention may be selected from a single chain binding molecule which comprises an antigen binding site comprising
a) a first domain comprising in sequence the hypervariable regions CDR1, CDR2 and CDR3, said hypervariable regions having the amino acid sequences as shown in Seq. Id. No. 1 in EP 449,769, the contents of which is herein incorporated by reference,
b) a second domain comprising in sequence the hypervariable regions CDR1′, CDR2′ and CDR3′, said hypervariable regions having the amino acid sequences as shown in Seq. Id. No. 2 in EP 449,769, the contents of which is herein incorporated by reference, and
c) a peptide linker which is bound either to the N-terminal extremity of the first domain and to the C-terminal extremity of the second domain or to the C-terminal extremity of the first domain and to the N-terminal extremity of second domain;
   and direct equivalents thereof.

As it is well known, minor changes in an amino acid sequence such as deletion, addition or substitution of one, more or several amino acids may lead to an aIlelic form of the original protein which has identical or substantially identical properties, e.g. antigen binding properties. Thus, by the term “direct equivalents thereof” is meant either any single domain CD25 binding molecule (molecule X)
(i) in which the hypervariable regions CDR1, CDR2 and CDR3 taken as a whole are at least 80% homologous, preferably at least 90% homologous, more preferably at least 95% homologous to the hypervariable regions as shown in Seq. Id. No. 1 in EP 449,769, the contents of which is herein incorporated by reference, and,
(ii) which is capable of inhibiting the binding of Interleukin 2 (IL-2) to its receptor substantially to the same extent as a reference molecule having framework regions identical to those of molecule X but having hypervariable regions CDR1, CDR2 and CDR3 identical to those shown in Seq. Id. No. 1 in EP 449,769, the contents of which is herein incorporated by reference;
   or any CD25 binding molecule having at least two domains per binding site (molecule X′)
(i) in which the hypervariable regions CDR1, CDR2, CDR3, CDR1′, CDR2′ and CDR3′ taken as a whole are at least 80% homologous, preferably at least 90% homologous, more preferably at least 95% homologous to the hypervariable regions as shown in Seq. Id. No. 1 and 2 in EP 449,769, the contents of which is herein incorporated by reference, and
(ii) which is capable of inhibiting the binding of IL-2 to its receptor substantially to the same extent as a reference molecule having framework regions and constant parts identical to molecule X′ but having hypervariable regions CDR1, CDR2, CDR3, CDR1′, CDR2′ and CDR3′ identical to those shown in Seq. Id. No. 1 and 2 in EP 449,769, the contents of which is herein incorporated by reference.

This last criterion may be conveniently tested in various assays as described in EP 449,769, the contents of which is herein incorporated by reference.

A most preferred CD25 binding molecule for use in accordance with the invention is a chimeric CD25 antibody, especially a chimeric CD25 antibody comprising at least
a) one heavy chain which comprises a variable domain having an amino acid sequence identical or substantially identical to that shown in Seq. Id. No. 1 in EP 449,769, the contents of which is herein incorporated by reference, starting with amino acid at position 1 and ending with amino acid at position 117 and the constant part of a human heavy chain; and
b) one light chain which comprises a variable domain having an amino acid sequence identical or substantially identical to that shown in Seq. Id. No. 2 in EP 449,769, the contents of which is herein incorporated by reference, starting with glutamic acid at position 1 and ending with glutamic acid at position 104 and the constant part of a human light chain.

The constant part of a human heavy chain may be of the γ1, γ2, γ3, γ4, μ, α1, α2, δ or ε type, preferably of the γ type, more preferably of the γ1 type, whereas the constant part of a human light chain may be of the κ or λ type (which includes the λ1, μ2 and λ3 subtypes) but is preferably of the κ type. The amino acid sequence of all these constant parts are given in Kabat et al., Sequences of Proteins of Immunological Interest, US Department of Health and Human Services, Public Health Service, NIH.

The most preferred CD25 binding molecule is basiliximab which is commercially available as SIMULECT® from Novartis AG.

The CD25 binding molecules suitable for use in accordance with the present invention may be produced by techniques disclosed for example in EP 449,769, the contents of which is herein incorporated by reference, in particular in Examples 1 to 5 of EP 449,769.

As described in EP-B-449,769, the CD25 binding molecules have, on the basis of observed activity in e.g. a Mixed Lymphocyte Reaction assay, been found to be useful for preventing or treating graft rejection episodes.

In accordance with the present invention it has now surprisingly been found that the CD25 binding molecules are effective in the treatment of inflammatory diseases of the gastro-intestinal tract.

Therefore the invention also provides
(i) A method for the treatment of an inflammatory disease of the GI tract in a patient in need of such treatment comprising administering to the patient an effective amount of a CD25 binding molecule as described above.
(ii) A method for the treatment of inflammatory disease of the GI tract in a subject in need of such treatment comprising administering, e.g. concomitantly or in sequence, to said subject an effective amount of a) a CD25 binding molecule as described above and b) a further drug substance being effective in the treatment of inflammatory diseases of the gastro-intestinal tract.
(iii) A pharmaceutical composition for use in a method as described in (i) to (ii) which comprises a CD25 binding molecule as described above and a pharmaceutically acceptable carrier or diluent.
(iv) A CD25 binding molecule as described above for use in the manufacture of a medicament for use in a method as described in (i) or (ii).
(v) A therapeutic combination, e.g. a kit or package, for use in any of the methods as described in (i) or (ii) said combination including a pharmaceutical composition comprising a CD25 binding molecule as described above, and further including at least one pharmaceutical composition comprising a further drug substance effective in the treatment of inflammatory diseases of the GI tract.

For the use in accordance with the invention, the appropriate dosage will, of course, vary depending upon, for example, the particular CD25 binding molecule to be employed, the host, the mode of administration and the severity of the condition being treated and the effects desired. Satisfactory results are generally indicated to be obtained at dosages from about 0.1 mg to about 100 mg. Administration may be in a single dose or in several doses over a period of time as long as may be indicated in relation to the time the disease is clinically evident or prophylactically to suppress further clinical relapse, for example a dose from about 5 up to about 100 mg may be administered with a time-lag from 1 day up to five weeks, e.g. every 3 to 6 days, until control or amelioration of the disease is achieved. A preferred dosage regimen comprises administration of 20 mg of CD 25 binding molecule, e.g. basiliximab, on day 0 and administration of a further 20 mg on day 4.The CD25 binding molecule is conveniently administered parenterally, e.g. intravenously, for example, into the antecubital or other peripheral vein. An alternative exemplary dosing regimen is intravenous administration of 40 mg every 28 days until control or amelioration of the disease is achieved.

Pharmaceutical compositions of the invention may be manufactured in a conventional manner as described, e.g. in EP 449,769, the contents of which is herein incorporated by reference.

The CD25 binding molecule may be administered as the sole active ingredient or together with other drugs in immunomodulating regimens or other anti-inflammatory agents. For example, the CD25 binding molecule may be used in accordance with the invention in combination with cyclosporins, rapamycins or ascomycins, or their immunosuppressive analogs, e.g. cyclosporin A, cyclosporin G, FK-506, rapamycin etc.; corticosteroids e.g. prednisone; cyclophosphamide; azathioprene; methotrexate; gold salts, sulfasalazine, antimalarials, brequinar; leflunomide; mizoribine; mycophenolic acid; mycophenolate mofetil; 15-deoxyspergualine; other immuno-suppressive monoclonal antibodies, e.g. monoclonal antibodies to leukocyte receptors, e.g. MHC, CD2, CD3, CD4, CD7, CD28, B7, CD40, CD45, or CD58 or their ligands; or other immunomodulatory compounds, e.g. CTLA4lg.

If the CD25 binding molecule is co-administered with a further drug substance both may be packaged separately within the same container, with instructions for mixing or concomitant administration. Examples of kits include for example a multi-barreIled syringe or a twin pack containing separate unit dose forms.

The intravenous infusions may be prepared as follows: the lyophylized antibodies are mixed together and dispersed into 100 ml sterile buffered saline containing 4.5% wt. of human albumin. This saline dispersion may be administered to the patients either as an intravenous bolus injection or as an intravenous infusion over a 15 minute period.

Investigations so far indicate that the administration of the CD25 binding molecules is free from unacceptable side-effects at the dosage levels employed. Particularly the preferred one, basiliximab, is safe, approved by the Federal Drug Administration (FDA) of the United States and is commercially available.

The utility of the CD 25 binding molecules for the treatment of inflammatory diseases of the GI tract may be assessed in various animal model systems as well as in clinical trials, for instance as hereinafter described. Thus, for example, the ability of CD 25 binding molecules to reduce colonic inflammation is demonstrated using the trinitrobenzene sulphonic acid (“TNBS”) model for IBD.

Rat TNBS model

The TNBS model is one of the standard IBD models used in IBD discovery research and it has been extensively evaluated in rodents. See, for example, C. O. Elson et al. (1995), Experimental Models of Inflammatory Bowel Disease, Gastroenterology, 109: 1344-1367 and references cited therein. In this model, asingle enema of TNBS induces a prolonged colonic inflammatory response (up to several weeks) that is transmural and is accompanied by oxidative damage as evidenced by an increase in myeloperoxidase (“MPO”) activity. Additionally, the inflammation is characterized by discrete areas of acute necrosis, inflammation and muscle thickening. Agents with anti-inflammatory effects in patients with IBD show efficacy in this model. Although the mechanism by which TNBS induces an inflammatory response is unknown, it is thought to have an immunological basis.

Induction of Colitis

Male Sprague-Dawley rats (200-250 g) are housed in standard cages (2 per cage) and fed rat chow and tap water ad libitum. After an overnight fast, rats are brought into the laboratory and randomized into treatment groups. Colitis is induced by intrarectal administration of 0.5 ml of TNBS solution (50 mg/kg in 50% ethanol) using a 1 mL syringe attached to a 5 cm polyethylene catheter. Control animals received saline (0.9%) or a 1% methyl cellulose suspension at identical time points.

Tissue Analyses

days after TNBS administration, the rats are sacrificed and the colons excised and opened longitudinally. In 5 cm segments of colon, gross morphology is determined using the following scale:

| Grade | Finding |
|---|---|
| 0 | No damage |
| 1 | One area of Inflammation (red), no ulcers |
| 2 | Ulcers, no area of inflammation |
| 3 | Ulcers, one area of inflammation |
| 4 | More than 2 ulcers, inflammation at one site |
| 5 | More than 2 ulcers, inflammation > 1 cm |

The weights of each 5 cm colonic segment are also recorded to assess inflammatory induced edema.

Dosing Regimen

CD-25 binding molecules are tested in the TNBS model at 1 mg/kg s.c. (oral) dosing. Each of the test compounds is administered by s.c. injection 1 hour prior to the administration of TNBS. Control rats are given saline only.

CD-25 binding molecules typically reduce TNBS-induced damage compared to the controls. Alternative animal model systems which may be used to evaluate the ability of CD 25 molecules to reduce colonic inflammation, include the mouse dextran sulfate IBD Model and other models described in Elson et al. (ibid).

Crohn's Disease Trial

Utility of the CD25 binding molecule for the treatment of inflammatory diseases of the GI tract is shown in the following clinical example which describes the use of basiliximab in the maintenance of remission of Crohn's Disease.

60 Crohn's Disease patients are enroIled for the trial. Eligibility criteria include age of at least 18 years, a history compatible with Crohn's Disease confirmed by either a typical barium X-ray examination, gross appearance at the time of surgery, endoscopy, or pathology, including histology.

Crohn's Disease (including histology)

Participants are those who report at least two flare-ups of active disease within the last 4 years, one within the last 18 months or a recent resection. Remission is defined as a Crohn's Disease Activity Index (CDAI) (Best et al. Gastroenterology 1976; 70:439-444) score of <150 at baseline with no symptoms for the previous 30 days.

Exclusion criteria include a previous total proctocolectomy; short-bowel syndrome; a history of more than three resections within the last 10 years; chronic perianal disease that might interfere with assessments; a diagnosis of ulcerative colitis; stools positive for pathogens (Salmonella, Shigella, Yersinia and Campylobacter), parasites, or Clostridium difficile toxin; a history of alcohol or drug abuse; clinically significant hepatic, neurological, endocrine, renal, or other major systemic disease that would make implementation or interpretation of the protocol or results difficult; any history of cancer (excluding basal cell or squamous cell carcinoma of the skin); and inability to give informed consent. Patients are also excluded if they have taken immunosuppressive drugs (azathioprene, 6-mercaptopurine, and cyclosporine) within the last 90 days, corticosteroids within the last 30 days, or mesalamine or metronidazole within the last 7 days.

During the study the following medicaments are specifically excluded: oral or rectal corticosteroids; mesalamine preparations; aspirin or other nonsteroidal anti-inflammatory drugs, immunosuppressive drugs; narcotics aside from codeine to loperamide, which are permitted for the control of diarrhea; long term (>4 weeks) use of cholestyramine; sucralfate; H₂-blockers; and omeprazole or antacids and antibiotics for duration of >14 days.

The 60 patients are randomized to one of two groups; those receiving basiliximab and those receiving placebo.

Before entry a screening visit is carried out, at which informed consent is obtained and a diary card for calculation of CDAI is dispensed. The patient's history of Crohn's Disease and general demographics are reviewed; and a stool specimen requested for ova, parasites, culture sensitivity and C. difficile determination. Patients with a CDAI <150 return for a baseline assessment consisting of a review of the diary card for the last 7 days and calculation of the CDAI. A physical examination is performed; patients complete a quality of life questionnaire, the Inflammatory Bowel Disease Questionnaire (IBDQ) (Guyatt et al., Gatroenterology; 1989; 96:804-810), and a variety of assessments including a complete blood count, erythrocyte sedimentation rate, serum biochemistry (glucose, urea, nitrogen, creatinine, electolytes, calcium, phosphorous, bilirubin, alkaline phosphatase, aspartate aminotransferase, lactate dehydrogenase, total protein, albumin, amylase, and lipase), as well as a urinalysis are performed.

Basiliximab is administered intravenously at a dose of 60 mg on Day 0, 40 mg on Day 90 and 40 mg on Day 180. Patients are evaluated at Weeks 4, 12, 24, 36 and 48 for safety, efficacy and disease outcome. Each assessment consists of a physician's review, including physical examination, a quality of life assessment, repeat laboratory investigations, and an enquiry into adverse events. At each visit an evaluation of abdominal pain, presence of abdominal tenderness or mass, functional capacity, and nutritional status is performed. On each occasion the CDAI is calculated.

The primary efficacy outcome measures are the relapse time and time to relapse. Treatment failure or relapse is defined as the first occurence of a CDAI that is >150 as well as the absolute figure being at least 60 points higher than base line.

Patients receiving basiliximab show a clear maintenance of remission of Crohn's Disease as compared to patients receiving placebo.

Ulcerative Colitis Trial

Utility of the C 25 binding molecules for the treatment of ulcerative is shown in a clinical trial similar to that described above for Crohn's disease. Patients diagnosed as suffering from ulcerative colitis are the subjects of such a trial and the Rachmillewitz index is used for scoring disease severity. Exclusion criteria include a Rachmillewitz index of <6, and other criteria as described for Crohn's disease. Similar treatment regimes are used as for Crohn's disease. Patients receiving CD 25 binding molecules, e.g. basiliximab show amelioration of symptoms as compared to patients receiving placebo.

1. A CD25 binding molecule which comprises at least one antigen binding site comprising at least one domain which comprises in sequence, the hypervariable regions CDR1, CDR2 and CDR3; said CDR1 having the amino acid sequence Arg-Tyr-Trp-Met-His (SEQ ID NO:1), said CDR2 having the amino acid sequence Ala-Ile-Tyr-Pro-Gly-Asn-Ser-Asp-Thr-Ser-Tyr-Asn-Gln-Lys-Phe-Glu-Gly (SEQ ID NO:2), and said CDR3 having the amino acid sequence Asp-Tyr-Gly-Tyr-Tyr-Phe-Asp-Phe (SEQ ID NO:3); or direct equivalents thereof, for use in the treatment of inflammatory disease of the gastro-intestinal tract.

2. A CD25 binding molecule as defined in claim 1, for use in the manufacturing of a medicament for use in the treatment of inflammatory disease of the gastro-intestinal tract.

3. A pharmaceutical composition for the treatment of inflammatory disease of the gastro-intestinal tract comprising a CD25 binding molecule as defined in claim 1 and a pharmaceutically acceptable carrier or diluent.

4. A method for the treatment of inflammatory disease of the gastro-intestinal tract in a patient in need of such treatment comprising administering to the patient an effective amount of a CD25 binding molecule as defined in claim 1.

5. A method for the treatment of inflammatory disease of the gastro-intestinal tract in a subject in need of such treatment comprising administrating to said subject an effective amount of a) a CD25 binding molecule as defined in claim 1 and b) a further drug substance being effective in the treatment of inflammatory disease of the gastro-intestinal tract.

6. A therapeutic combination for use in a method as described in claim 5 said combination including a pharmaceutical composition comprising a CD25 binding molecule as defined in claim 1, and further including at least one pharmaceutical composition comprising a further drug substance effective in the treatment of inflammatory disease of the gastro-intestinal tract.

7. A method according to any one of claims 4 to 5, wherein the CD25 binding molecule is basiliximab.

8. A CD25 binding molecule according to claim 1, which is basiliximab.

9. A composition or combination according to claim 3, wherein the CD25 binding molecule is basiliximab.

10. A CD25 binding molecule according to claim 1 for use in the treatment of Irritable Bowel Syndrome (IBS), Crohn's Disease, ulcerative colitis or inflammatory intestinal disease.
